(19) United States
(12) Patent Application Publication
(10) Pub. No.: US 2024/0115425 A1
(43) Pub. Date: Apr. 11, 2024

(54) EAR COVERING DEVICE

(71) Applicant: Wade David Pye, Scarborough (CA)

(72) Inventor: Wade David Pye, Scarborough (CA)

(21) Appl. No.: 17/938,356

(22) Filed: Oct. 6, 2022

(51) Int. Cl. A61F 11/14

(52) U.S. Cl. CPC A61F 11/14

(57) ABSTRACT

An ear covering device that is configured to be releasably secured to the head of a user that includes a first ear covering member and a second ear covering member. A securing member is provided that is configured to be superposed the top of a user's head and includes a first end and a second end. The first ear covering member and the second ear covering member include slots that are operable to receive the first end and second end of the securing member. The first ear covering member and second ear covering member each include an inner surface and an outer surface that are axially parallel. In a preferred embodiment the first ear covering member and the second ear covering member are formed in the shape of a hockey puck. Marketing indicia is present on the outer surface of the first ear covering member and second ear covering member.

### FIELD OF THE INVENTION

The present invention relates generally to headwear accessories, more specifically but not by way of limitation, an ear covering device that is configured to cover both ears and have a securing member on the head wherein the ear members have a unique novelty form.

### BACKGROUND

There are numerous types of ear coverings available on the marketplace. Ear coverings range from those types that solely secure around the ear themselves to conventional ear muffs and other devices. It is quite common to combine articles of clothing with marketing. This can be as simple as placing a brand name or logo on an article of clothing or can be more expansive wherein the article of clothing has a specific shape or design to make a more impactful marketing statement. Examples can include hats that resemble football helmets or novelty gloves that are utilized by fans attending sport events. While these conventional items exist, there are numerous opportunities for unique items such as but not limited to wearable accessories that are currently not available in the marketplace.

Accordingly, there is a need for an ear covering device that meets a unique marketing need by delivering a specific shape for the ear members thereof.

### SUMMARY OF THE INVENTION

It is the object of the present invention to provide an ear covering device that is configured to be releasably secured to a user's head wherein the present invention includes a securing member.

Another object of the present invention is to provide a headwear accessory that can be utilized for marketing purposes that is operable to be releasably secured to a user's head wherein the securing member is manufactured from a resilient material and is arcuate in shape having a first end and a second end.

A further object of the present invention is to provide an ear covering device that is configured to be releasably secured to a user's head that includes a first ear covering member and a second ear covering member.

Still another object of the present invention is to provide a headwear accessory that can be utilized for marketing purposes that is operable to be releasably secured to a user's head wherein the first ear covering member and the second ear covering member are secured to the first end and second end of the securing member respectively.

An additional object of the present invention is to provide an ear covering device that is configured to be releasably secured to a user's head wherein the first ear covering member and second ear covering member in a preferred embodiment are formed in the shape of a hockey puck having an inner surface and outer surface that are planar in manner.

Yet a further object of the present invention is to provide a headwear accessory that can be utilized for marketing purposes that is operable to be releasably secured to a user's head wherein the first ear covering member and second ear covering member include a slot formed therein to receive an end of the securing member.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a side perspective view of the present invention; and

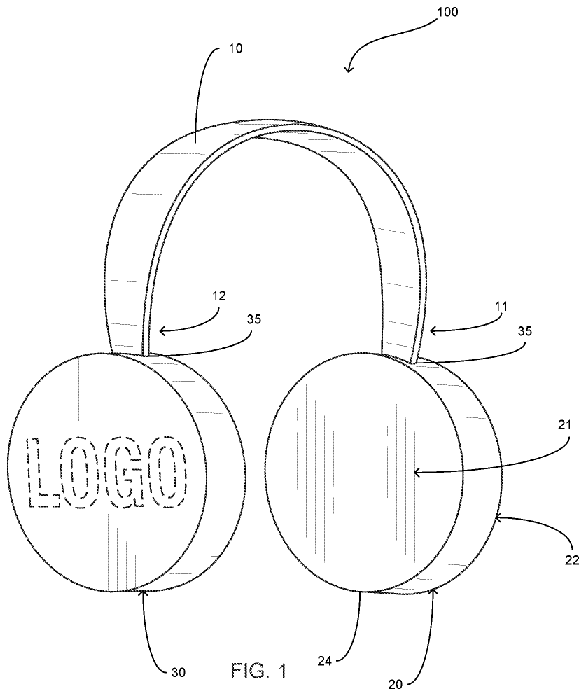

FIG. 2 is a side perspective view of the present invention on a user's head; and

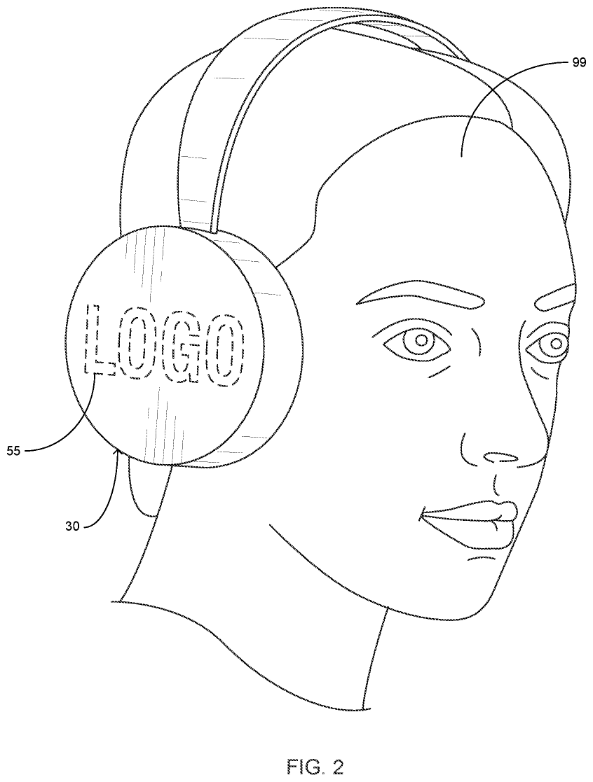

### DETAILED DESCRIPTION

Referring now to the drawings submitted herewith, wherein various elements depicted therein are not necessarily drawn to scale and wherein through the views and figures like elements are referenced with identical reference numerals, there is illustrated an ear covering device 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular the Figures submitted as a part hereof, the ear covering device 100 includes a securing member 10 that is manufactured from a resilient material such as but not limited to plastic and is arcuate in shape. The securing member 10 functions to releasably secure the ear covering device 100 to the head 99 of the user by extending over the top portion thereof. The securing member 10 includes a first end 11 and a second end 12. While a preferred embodiment of the securing member 10 is illustrated herein, it should be understood that alternate embodiments of the securing member 10 could be provided within the scope of the present invention to achieve the discussed objective herein.

The ear covering device 100 includes a first ear covering member 20 and a second ear covering member 30. The first ear covering member 20 and second ear covering member 30 are identically constructed and as such the ensuing discussion of the elements of the first ear covering member 20 should be understood to apply to the second ear covering member 30. The first ear covering member 20 includes an inner surface 21 and an outer surface 22. The inner surface 21 and outer surface 22 are both planar in manner and are axially parallel. Both the inner surface 21 and outer surface 22 are solid having no depressions or formations and are planar (or flat) within the perimeter edge 24 of the first ear covering member 20. The firs ear covering member 20 has an outer surface that is manufactured from a suitable material such as but not limited to vinyl. The interior of the first ear covering member 20 (not particularly illustrated herein) is manufactured from a foam rubber or other suitable material that will not provide discomfort when the ear covering device 100 is in place on the head 99.

The first ear covering member 20 includes a slot 35 formed therein wherein the slot 35 is configured to receive therein the end 11 so as to releasably secure the first ear covering member 20 thereto. The end 11 is journaled into the slot 35 sufficiently so as to maintain engagement therewith. Releasably securing the first ear covering member 20 provides the ability for the first ear covering member 20 to be removed for cleaning or exchanged for an alternate ear covering member. The first ear covering member 20 includes indicia 55 imprinted on the outer surface 22 thereof. In a preferred embodiment of the present invention the first ear covering member 20 the first ear covering member 20 is shaped similarly to a hockey puck. The indicia 55 can be a team logo, player name or similar marketing indicia related to a desired team. While a hockey puck shape is the preferred embodiment for the first ear covering member 20 it should be understood that the first ear covering member 20 could be provided in alternate shapes or resemble alternate sports elements such as but not limited to a baseball or football. It should be understood within the scope of the present invention that any alternative embodiment of the first ear covering member 20 that the inner surface 21 and outer surface 22 will be planar (flat) and axially parallel.

While not specifically illustrated herein, it is contemplated within the scope of the present invention that the inner surface 21 of the first ear covering member 20 could have releasably secured thereto a liner wherein the liner is configured to provide warmth for the ear of the user of the ear covering device 100. Those skilled in the art will recognize that the liner could be manufactured from various suitable materials.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. An ear covering device that is configured to cover ears of a user wherein the ear covering device comprises:
a securing member, said securing member having a first end and a second end, said securing member being arcuate in shape, said securing member configured to be superposed a top of a user's head;
a first ear cover member, said first ear cover member being releasably secured to said first end of said securing member, said first ear cover member having an inner surface and an outer surface, said first ear cover member being adjacent an ear of a user, said inner surface and said outer surface being flat in manner and being axially parallel; and
a second ear cover member, said second ear cover member being releasably secured to said second end of said securing member, said second ear cover member having an inner surface and an outer surface, said second ear cover member being adjacent an ear of a user, said inner surface and said outer surface of said second ear cover member being flat in manner and being axially parallel.

2. The ear covering device that is configured to cover ears of the user as recited in claim 1, wherein said first ear cover member and said second ear cover member are annular in shape.

3. The ear covering device that is configured to cover ears of the user as recited in claim 2, wherein said first ear cover member and said second ear cover member have a vinyl outer covering.

4. The ear covering device that is configured to cover ears of the user as recited in claim 3, wherein said first ear cover member and said second ear cover member are formed to resemble a hockey puck.

5. The ear covering device that is configured to cover ears of the user as recited in claim 3, wherein said first ear cover member and said second ear cover member are formed to resemble a sports ball.
